SEQUENCE LISTING (44 sequences)

SEQ ID NO: 1 - DNA, 30 bp, Artificial Sequence
  Nucleotide Primer/Probe
caatgtagaacattttagttcacgaaatgt

SEQ ID NO: 2 - DNA, 30 bp, Artificial Sequence
  Nucleotide Primer/Probe
caatgtagaacattttagttcacgaaatga

SEQ ID NO: 3 - DNA, 24 bp, Artificial Sequence
  Nucleotide Primer/Probe
tggtatttacagcatcccgccgta

SEQ ID NO: 4 - DNA, 21 bp, Artificial Sequence
  Nucleotide Primer/Probe
attttccaattttgcgtcaga

SEQ ID NO: 5 - DNA, 20 bp, Artificial Sequence
  Nucleotide Primer/Probe
ttgggagtgagatgtctgga

SEQ ID NO: 6 - DNA, 15 bp, Artificial Sequence
  Nucleotide Primer/Probe
gagctgctctgccat

SEQ ID NO: 7 - DNA, 15 bp, Artificial Sequence
  Nucleotide Primer/Probe
gagctgctctgcaat

SEQ ID NO: 8 - DNA, 26 bp, Artificial Sequence
  Nucleotide Primer/Probe
ctagactagtggagactaaaaatggt

SEQ ID NO: 9 - DNA, 26 bp, Artificial Sequence
  Nucleotide Primer/Probe
ctagactagtggagactaaaaatggg

SEQ ID NO: 10 - DNA, 30 bp, Artificial Sequence
  Nucleotide Primer/Probe
ctaggactaaagtgacaacaaatcacaatt

SEQ ID NO: 11 - DNA, 24 bp, Artificial Sequence
  Nucleotide Primer/Probe
caacgtattctcgaactaaaatgg

SEQ ID NO: 12 - DNA, 28 bp, Artificial Sequence
  Nucleotide Primer/Probe
ttgaattaagatcaaagagaagaattga

SEQ ID NO: 13 - DNA, 13 bp, Artificial Sequence
  Nucleotide Primer/Probe
cacccacttgatc

SEQ ID NO: 14 - DNA, 14 bp, Artificial Sequence
  Nucleotide Primer/Probe
cccacttgttcatc

SEQ ID NO: 15 - DNA, 27 bp, Artificial Sequence
  Nucleotide Primer/Probe
cataaacacctccatttccaaaagcat

SEQ ID NO: 16 - DNA, 26 bp, Artificial Sequence
  Nucleotide Primer/Probe
ataaacacctccatttccaaaagcac

SEQ ID NO: 17 - DNA, 25 bp, Artificial Sequence
  Nucleotide Primer/Probe
gtagagtgttccttcgcatgtggat

SEQ ID NO: 18 - DNA, 28 bp, Artificial Sequence
  Nucleotide Primer/Probe
ggacctttaatgtcatgtaaaagatcct

SEQ ID NO: 19 - DNA, 27 bp, Artificial Sequence
  Nucleotide Primer/Probe
gacctttaatgtcatgtaaaagatccc

SEQ ID NO: 20 - DNA, 30 bp, Artificial Sequence
  Nucleotide Primer/Probe
gcacaaaatatgcataaaagtaatgaggat

SEQ ID NO: 21 - DNA, 21 bp, Artificial Sequence
  Nucleotide Primer/Probe
cccaagtgaaggctgaaccag

SEQ ID NO: 22 - DNA, 23 bp, Artificial Sequence
  Nucleotide Primer/Probe
atcccaagtgaaggctgaaccaa

SEQ ID NO: 23 - DNA, 24 bp, Artificial Sequence
  Nucleotide Primer/Probe
gttgttaggccacgccctacactt

SEQ ID NO: 24 - DNA, 29 bp, Artificial Sequence
  Nucleotide Primer/Probe
tagccaatttttgtgtcaacattttgtag

SEQ ID NO: 25 - DNA, 28 bp, Artificial Sequence
  Nucleotide Primer/Probe
agccaatttttgtgtcaacattttgtac

SEQ ID NO: 26 - DNA, 29 bp, Artificial Sequence
  Nucleotide Primer/Probe
caaggcaattgttgagtccgtcaaattaa

SEQ ID NO: 27 - DNA, 21 bp, Artificial Sequence
  Nucleotide Primer/Probe
gaaggtgaccaagttcatgct

SEQ ID NO: 28 - DNA, 21 bp, Artificial Sequence
  Nucleotide Primer/Probe
gaaggtcggagtcaacggatt

SEQ ID NO: 29 - DNA, 401 bp, Glycine max
ataaaaatactttctaatgtagtagacataggcatattcaatcacattcttttaaattcaacagtgaataacagcccggtagactgtagtattttttattgaaattataactatgaaaaaatgtaaatactagaactcaaaggtagctcctggtatttacagcatcccgccgtacatatagatctctctcgctctctctcwcatttcgtgaactaaaatgttctacattgacaatggcatcaaaactcaatagaaaaagaagaaaaaagygaacgaagtatgctcaacaacttgtgtctgatctgggtgtttcattgtattgtagaagagaaatctggcgctgcagcctcatttgctgatagaatttagcaatgaactcctcagcttgcttatctattccc

SEQ ID NO: 30 - DNA, 81 bp, Glycine max
tggtatttacagcatcccgccgtacatatagatctctctcgctctctctcwcatttcgtgaactaaaatgttctacattga

SEQ ID NO: 31 - DNA, 604 bp, Glycine max
gtgtttgctacttgcagattcttctgagcatgtttgcatttacaacagcaatattagcactggtcatgatgcaattgtgctgaagagtggttgggatcaatatggagttgcctatggcaaaccaacctcaaaggtccacatccggggtgtttatctacaatcttcatctggcgctggcctagcatttgggagtgagatgtctggaggcatatctgatataatkgcagagcagctccatattaccaactcaactattggcattgaactgaagacaactargggtagaggtggttatatgaaaaacatcttcatttctgacgcaaaattggaaaatatttacttgggtatcagcatgacgggaagctctggctcccatccagatgacaagtacgatcctaatgcagttccggatgttggtaatgttacttttgagaatgtgattggtacaaatattgctattgctgggaatttttcaggaatagttgattcgcctttcactcctatctgtcttttgaacgcgaccttttcttccagttctgagtcatcatctccttcatggttttgttctgacataatgggaatctccgaggaagtcatggtcata

SEQ ID NO: 32 - DNA, 157 bp, Glycine max
gcctagcatttgggagtgagatgtctggaggcatatctgatataatkgcagagcagctccatattaccaactcaactattggcattgaactgaagacaactargggtagaggtggttatatgaaaaacatcttcatttctgacgcaaaattggaaaa

SEQ ID NO: 33 - DNA, 401 bp, Glycine max
actctggttatggtatgtaactttttctaacattaagtgtgtttttgctaattgcaaatgaacctgtggtcatcaaacatttaaaaccttgtaggattaaactgagttgcatttcaattggaaagatcaatgtaatagatgtgccatgctattagtttaggctgaacaagcttttctagactagtggagactaaaaatggkcatggaartgaaagagtaaatgatcaatttagttcctgtaattgtgatttgttgtcactttagtcctagaattaactaaattcaaacagtccctgaaactgtgatccgtcacccaaaagttatatgcaacggaacatgtgacttaaatgaacatataacatgttaacacatgtccagggaccaaattggcagttagtggt

SEQ ID NO: 34 - DNA, 96 bp, Glycine max
tctagactagtggagactaaaaatggkcatggaartgaaagagtaaatgatcaatttagttcctgtaattgtgatttgttgtcactttagtcctag

SEQ ID NO: 35 - DNA, 778 bp, Glycine max
ttcttttggtggtatgtcgaactgtaattttactagatatattcaaccgtgtttgaattccaatttttctttcgatgaattttatctatttattcattacacttgttgattatgttatgtcgtctccgcccctaattggcagatccaacagtcaagaakatcatgactcgggcaagcattactgcgtgaggtcgtaatatgggaaattatattcaaagtacttttgttgttaaaagagtagtcatatttggacatcctaatattttttatatctctcatttattttggtttctctctttttattatatcaaatatatattatatttatcatttttttttctctaagtattaaatattaatgtacagaatgtccatgaaacattttcctttttgttatgcgccggaaaagaacatgcggacatgcaattatcttttgacaaaattcgcaaatgtttaaacaattatcttgacaaaatttgcaaatgttgaaatttaaaatattttgtttgaattaagatcaaagagaagaattgataatttgtttggtgttgtaactttttattaaagaagtttttctttggatgawcaagtgggtgggtcaaagacattctcttttaataaaaaaatgttaaccattttagttcgagaatacgttggggggatcttgtcgtgaaaaacgtaataatcctttggatgattgaggcgatgagacgccaaagcagttaatagacgcatatggttagaataaaaagaatccagtggcgcagcgcagctggtt

SEQ ID NO: 36 - DNA, 150 bp, Glycine max
ttgaattaagatcaaagagaagaattgataatttgtttggtgttgtaactttttattaaagaagtttttctttggatgawcaagtgggtgggtcaaagacattctcttttaataaaaaaatgttaaccattttagttcgagaatacgttg

SEQ ID NO: 37 - DNA, 401 bp, Glycine max
gggttgaggtttttgagcatctttttgaggccgctgcagttgtaggacgttgatcttacctcgtacatggtatggttgaccactgaggatatcacttgtgggacagattttgttgtgagatctgggaatgtggctagtgttaagacacataggtctgggtagagtgttccttcgcatgtggattgggctactacttgtatrtgcttttggaaatggaggtgtttatgtttttgatctgggactcttgttgtttgggtgttggttaaatgagttgttccaaagaatagtagagaagaaatcacggtcagaatggatgcaagtgtgaggttgttcatgatgtgataggacttaggaggtgggttaggattactgtgtatgtgtaactagctagcagggtacgt

SEQ ID NO: 38 - DNA, 69 bp, Glycine max
gtagagtgttccttcgcatgtggattgggctactacttgtatrtgcttttggaaatggaggtgtttatg

SEQ ID NO: 39 - DNA, 401 bp, Glycine max
gttgattttttgtctttattaatttgaatttttaaaaattattttgagctaatcctcaagacctaaattcctcgtataatggatttttgcaaatgatccataaggctcgagtgacacaacctgacacaaactaattgaacaatgaaataagaaggcacaaaatatgcataaaagtaatgaggatamaatcagcaaaacagrggatcttttacatgacattaaaggtcctgtaagaaggtctaatactatgagggtgaaataagatttgcaagatgtaattacgaatatcaaagaaagtatatataagtcaaagtatgttaaaggcaacacaaaaactagtcaatcttccacaagttaataatgatttgagtccaactttatgaaaacaatttggagagttt

SEQ ID NO: 40 - DNA, 74 bp, Glycine max
gcacaaaatatgcataaaagtaatgaggatamaatcagcaaaacagrggatcttttacatgacattaaaggtcc

SEQ ID NO: 41 - DNA, 401 bp, Glycine max
tctcttgcggttaaatctgaattccttctttgaggtaaaacaatcagatagataattagtctcactgtttaactttagatcaatgtttcttattgttgtttgttattttgtcccttaacaggcaactgctgcaaggggggttctgaatgttgttaggccacgccctacacttcctgtttaacttdagatcattagatttgytggttcagccttcacttgggatchbaaatttctactttttttttttggttaaatctggttctttttgttttctatcctcttcattgtctaaaaaatcaattttggttgtgattaaaattcaattttgtttttgtacggttctggatattgttctcttcatgttcttcttctgcaatgagattcatttggtttccctcttt

SEQ ID NO: 42 - DNA, 74 bp, Glycine max
gttgttaggccacgccctacacttcctgtttaacttdagatcattagatttgytggttcagccttcacttggga

SEQ ID NO: 43 - DNA, 401 bp, Glycine max
agctagtagcagagacaagaccatcaaattgtatactggaatgtgtcatggcataacaaccggagagacagatgaaaacattacacttgcgtttgttgacatcatagcttggttggataagcacgcaatcaaggcaattgttgagtccgtcaaattaatttaaagttatggcattgagacatagatcagaattgcattacstacaaaatgttgacacaaaaattggctaattgagttttgttatggccaacttcaccctccctggccccaatttgtttttccagaaatgaagcaaaatattttaggcttcaaaatcaaatattgtagtataattttcgtaacaacataaatattaggaaagattcgtagttacaacaatcttttattagtagtattccatt

SEQ ID NO: 44 - DNA, 101 bp, Glycine max
caaggcaattgttgagtccgtcaaattaatttaaagttatggcattgagacatagatcagaattgcattacstacaaaatgttgacacaaaaattggctaa

(19) United States
(12) Patent Application Publication
(10) Pub. No.: US 2013/0047301 A1
(43) Pub. Date: Feb. 21, 2013

(54) MOLECULAR MARKERS ASSOCIATED WITH SOYBEAN ROOT-KNOT NEMATODE TOLERANCE AND METHODS OF THEIR USE

(71) Applicants: Jennifer A. Klaiber, Urbandale, IA (US); Joshua M. Shendelman, Ankeny, IA (US); Mark J. Hood, Memphis, TN (US); Robert J. Suttner, Cordova, TN (US); Julian M. Chaky, Urbandale, IA (US)

(72) Inventors: Jennifer A. Klaiber, Urbandale, IA (US); Joshua M. Shendelman, Ankeny, IA (US); Mark J. Hood, Memphis, TN (US); Robert J. Suttner, Cordova, TN (US); Julian M. Chaky, Urbandale, IA (US)

(73) Assignee: Pioneer Hi-Bred International, Inc., Johnston, IA (US)

(21) Appl. No.: 13/585,956

(22) Filed: Aug. 15, 2012

Related U.S. Application Data
(60) Provisional application No. 61/523,982, filed on Aug. 16, 2011.

(51) Int. Cl. C12Q 1/68; C07H 21/04; A01H 5/00

(52) U.S. Cl. 800/312; 536/24.33; 536/24.3; 435/6.11

(57) ABSTRACT

This invention relates to molecular markers useful for identifying and, optionally, selecting soybean plants displaying tolerance, improved tolerance, or susceptibility to root-knot nematode, methods of their use, and compositions having one or more marker loci. In certain examples, the method comprises detecting at least one marker locus. In other examples, the method comprises detecting a haplotype comprising two or more marker loci. In further examples, the method further comprises crossing a selected soybean plant with a second soybean plant. This invention further relates to primers, probes, kits, systems, etc., useful for carrying out the methods described herein.

### CROSS-REFERENCE TO RELATED APPLICATIONS

This application claims priority to and benefit of U.S. Provisional Patent Application Ser. No. 61/523,982, filed on Aug. 16, 2011, which is hereby incorporated by reference in its entirety.

### FIELD OF THE INVENTION

This invention relates to molecular markers that are useful for identifying root-knot nematode tolerant or susceptible soybean plants and methods of their use.

### BACKGROUND

Soybeans (Glycine max L. Merr.) are a major cash crop and investment commodity in North America and elsewhere. Soybean oil is one of the most widely used edible oils, and soybeans are used worldwide both in animal feed and in human food production. Additionally, soybean utilization is expanding to industrial, manufacturing, and pharmaceutical applications. Soybeans are also vulnerable to more than one hundred different pathogens, with some pathogens having disastrous economic consequences. One important soybean pathogen is the root-knot nematode, which can severely impact yield.

Root-knot nematodes (Meloidogyne spp.) are a particular genera of plant-parasitic nematodes. They are distributed worldwide, and are obligate parasites of the roots of thousands of plant species, including soybean. Root-knot nematode infestation results in poor growth, reduced crop yield, and reduced resistance to secondary stressors, such as other diseases.

In some crops, genetic resistance or tolerance to root-knot nematodes has been found to exist. For instance, in soybean, root-knot nematode tolerance has been coarsely mapped to the top of linkage group (LG) O, more specifically to the marker interval flanked by Satt358 and Satt492 (Li et al. (2001) SSR mapping and confirmation of the QTL from P196354 conditioning soybean resistance to southern root-knot nematode. Theor Appl Genet 103:1167-1173).based on the public soybean map by Choi, et al., “A Soybean Transcript Map: Gene Distribution, Haplotype and Single-Nucleotide Polymorphism Analysis” (2007) Genetics 176:685-96

There remains a need for soybean plants with improved tolerance to root-knot nematode and methods for identifying and selecting such plants, including improved markers for identifying plants possessing tolerance or susceptibility.

### SUMMARY

This invention relates to molecular markers useful for identifying and, optionally, selecting soybean plants displaying tolerance, improved tolerance, or susceptibility to root-knot nematode, methods of their use, and compositions having one or more marker loci. In certain examples, the method comprises detecting at least one marker locus. In other examples, the method comprises detecting a haplotype comprising two or more marker loci. In further examples, the method further comprises crossing a selected soybean plant with a second soybean plant. This invention further relates to primers, probes, kits, systems, etc., useful for carrying out the methods described herein.

### SUMMARY OF THE SEQUENCES

SEQ ID NOs: 1-3 comprise nucleotide sequences of regions of the soybean genome, each capable of being used as a probe or primer, either alone or in combination, for the detection of marker locus S13251-1-K1 on LG-O. In certain examples, SEQ ID NOs: 1 and 2 are used as allele specific forward primers and SEQ ID NO: 3 is used as a common reverse primer. In other examples, each of SEQ ID NOs: 1 and 2 is used in conjunction with a 5′ nucleotide tail sequence, for example a tail sequence as shown in SEQ ID NO: 27 or 28.

SEQ ID NOs: 4-7 comprise nucleotide sequences of regions of the soybean genome, each capable of being used as a probe or primer, either alone or in combination, for the detection of marker locus S07191-1-Q2 on LG-O. In certain examples, SEQ ID NOs: 4 and 5 are used as primers while SEQ ID NOs: 6 and 7 are used as probes.

SEQ ID NOs: 8-10 comprise nucleotide sequences of regions of the soybean genome, each capable of being used as a probe or primer, either alone or in combination, for the detection of marker locus S14256-1-K1 on LG-O. In certain examples, SEQ ID NOs: 8 and 9 are used as allele specific forward primers and SEQ ID NO: 10 is used as a common reverse primer. In other examples, each of SEQ ID NOs: 8 and 9 is used in conjunction with a 5′ nucleotide tail sequence, for example a tail sequence as shown in SEQ ID NO: 27 or 28.

SEQ ID NOs: 11-14 comprise nucleotide sequences of regions of the soybean genome, each capable of being used as a probe or primer, either alone or in combination, for the detection of marker locus S07192-1-Q20 on LG-O. In certain examples, SEQ ID NOs: 11 and 12 are used as primers while SEQ ID NOs: 13 and 14 are used as probes.

SEQ ID NOs: 15-17 comprise nucleotide sequences of regions of the soybean genome, each capable of being used as a probe or primer, either alone or in combination, for the detection of marker locus S14242-1-K1 on LG-O. In certain examples, SEQ ID NOs: 15 and 16 are used as allele specific forward primers and SEQ ID. NO: 17 is used as a common reverse primer. In other examples, each of SEQ ID NOs: 15 and 16 is used in conjunction with a 5′ nucleotide tail sequence, for example a tail sequence as shown in SEQ ID NO: 27 or 28.

SEQ ID NOs: 18-20 comprise nucleotide sequences of regions of the soybean genome, each capable of being used as a probe or primer, either alone or in combination, for the detection of marker locus S13261-1-K1 on LG-O. In certain examples, SEQ ID NOs: 18 and 19 are used as allele specific forward primers and SEQ ID NO: 20 is used as a common reverse primer. In other examples, each of SEQ ID NOs: 18 and 19 is used in conjunction with a 5′ nucleotide tail sequence, for example a tail sequence as shown in SEQ ID NO: 27 or 28.

SEQ ID NOs: 21-23 comprise nucleotide sequences of regions of the soybean genome, each capable of being used as a probe or primer, either alone or in combination, for the detection of marker locus S14255-1-K1 on LG-O. In certain examples, SEQ ID NOs: 21 and 22 are used as allele specific forward primers and SEQ ID NO: 23 is used as a common reverse primer. In other examples, each of SEQ ID NOs: 21 and 22 is used in conjunction with a 5′ nucleotide tail sequence, for example a tail sequence as shown in SEQ ID NO: 27 or 28.

SEQ ID NOs: 24-26 comprise nucleotide sequences of regions of the soybean genome, each capable of being used as a probe or primer, either alone or in combination, for the detection of marker locus S14251-1-K1 on LG-O. In certain examples, SEQ ID NOs: 24 and 25 are used as allele specific forward primers and SEQ ID NO: 26 is used as a common reverse primer. In other examples, each of SEQ ID NOs: 24 and 25 is used in conjunction with a 5′ nucleotide tail sequence, for example a tail sequence as shown in SEQ ID NO: 27 or 28.

SEQ ID NOs: 27 and 28 comprise unique 5′ nucleotide tail sequences useful, for example, in KASPar real-time PCR detection.

SEQ ID NO: 29 is the genomic DNA region encompassing marker locus S13251-1-K1.

SEQ ID NO: 30 is the amplicon produced by amplifying genomic DNA using one or both of SEQ ID NOs: 1 and 2 as a forward or reverse primer in conjunction with SEQ ID NO: 3 as the other primer in the pair. This amplicon encompasses marker locus S13251-1-K1.

SEQ ID NO: 31 is the genomic DNA region encompassing marker locus S07191-1-Q2.

SEQ ID NO: 32 is the amplicon produced by amplifying genomic DNA using SEQ ID NO: 4 as a forward or reverse primer in conjunction with SEQ ID NO: 5 as the other primer in the pair. This amplicon encompasses marker locus S07191-1-Q2.

SEQ ID NO: 33 is the genomic DNA region encompassing marker locus S14256-1-K1.

SEQ ID NO: 34 is the amplicon produced by amplifying genomic DNA using one or both of SEQ ID NOs: 8 and 9 as a forward or reverse primer in conjunction with SEQ ID NO: 10 as the other primer in the pair. This amplicon encompasses marker locus S14256-1-K1.

SEQ ID NO: 35 is the genomic DNA region encompassing marker locus S07192-1-Q20.

SEQ ID NO: 36 is the amplicon produced by amplifying genomic DNA using SEQ ID NO: 11 as a forward or reverse primer in conjunction with SEQ ID NO: 12 as the other primer in the pair. This amplicon encompasses marker locus S07192-1-Q20.

SEQ ID NO: 37 is the genomic DNA region encompassing marker locus S14242-1-K1.

SEQ ID NO: 38 is the amplicon produced by amplifying genomic DNA using one or both of SEQ ID NOs: 15 and 16 as a forward or reverse primer in conjunction with SEQ ID NO: 17 as the other primer in the pair. This amplicon encompasses marker locus S14242-1-K1.

SEQ ID NO: 39 is the genomic DNA region encompassing marker locus S13261-1-K1.

SEQ ID NO: 40 is the amplicon produced by amplifying genomic DNA using one or both of SEQ ID NOs: 18 and 19 as a forward or reverse primer in conjunction with SEQ ID NO: 20 as the other primer in the pair. This amplicon encompasses marker locus S13261-1-K1.

SEQ ID NO: 41 is the genomic DNA region encompassing marker locus S14255-1-K1.

SEQ ID NO: 42 is the amplicon produced by amplifying genomic DNA using one or both of SEQ ID NOs: 21 and 22 as a forward or reverse primer in conjunction with SEQ ID NO: 23 as the other primer in the pair. This amplicon encompasses marker locus S14255-1-K1.

SEQ ID NO: 43 is the genomic DNA region encompassing marker locus S14251-1-K1.

SEQ ID NO: 44 is the amplicon produced by amplifying genomic DNA using one or both of SEQ ID NOs: 24 and 25 as a forward or reverse primer in conjunction with SEQ ID NO: 26 as the other primer in the pair. This amplicon encompasses marker locus S14251-1-K1.

### DETAILED DESCRIPTION

A novel method is provided for identifying a soybean plant or germplasm that displays tolerance, improved tolerance, or susceptibility to root-knot nematode, the method comprising detecting at least one allele of one or more marker loci associated with root-knot nematode tolerance.

In certain examples, the method involves detecting a single marker locus. In other examples, the method involves detecting a haplotype comprising two or more marker loci, for example, two marker loci, three marker loci, four marker loci, five marker loci, six marker loci, seven marker loci, eight marker loci, nine marker loci, 10 marker loci, 11 marker loci, 12 marker loci, 13 marker loci, 14 marker loci, 15 marker loci, 16 marker loci, 17 marker loci, 18 marker loci, 19 marker loci, 20 marker loci, or more. In certain examples, the haplotype comprises two or more markers selected from the group consisting of S13251-1-K1, S07191-1-Q2, S14256-1-K1, S07192-1-Q20, S14242-1-K1, S13261-1-K1, S14255-1-K1, and S14251-1-K1. In further examples, the haplotype comprises markers S07191-1-Q2, S14256-1-K1, and S07192-1-Q20.

In certain examples, the one or more alleles are favorable alleles that positively correlate with tolerance or improved tolerance to root-knot nematode. In other examples, the one or more alleles are disfavored alleles that positively correlate with susceptibility or increased susceptibility to root-knot nematode.

In certain examples, the one or more marker locus detected comprises one or more markers on LG-O selected from the group consisting of S13251-1-K1, S07191-1-Q2, S14256-1-K1, S07192-1-Q20, S14242-1-K1, S13261-1-K1, S14255-1-K1, and S14251-1-K1. In other examples, the one or more marker locus detected comprises one or more markers within the chromosome interval on linkage group O flanked by and including S13251-1-K1 and S14251-1-K1. In additional examples, the one or more marker locus detected comprises one or more markers within the chromosome interval on linkage group O flanked by and including S07191-1-Q2 and S07192-1-Q20. In further examples, the one or more marker locus detected comprises one or more markers within the chromosome interval on linkage group O flanked by and including Sat_{—}132 and Satt500. In other examples, the one or more marker locus detected comprises one or more markers within the chromosome interval on linkage group O flanked by and including Satt487 and Satt500. In still further examples, the one or more marker locus detected comprises one or more markers within the chromosome interval on chromosome 10 flanked by and including nucleotide positions 1338884 and 1593206. In yet further examples, the one or more marker locus detected comprises one or more markers within the chromosome interval on chromosome 10 flanked by and including nucleotide positions 1307986 and 1831976. In still further examples, the one or more marker locus detected comprises one or more markers within the chromosome interval on chromosome 10 flanked by and including nucleotide positions 1445721 and 1470496. In yet further examples, the one or more marker locus detected comprises one or more markers within one or more of the genomic DNA regions of SEQ ID NOs: 29, 31, 33, 35, 37, 39, 41, and 43. In other examples, the one or more marker locus detected comprises one or more markers within one or more of the amplicons of SEQ ID NOs: 30, 32, 34, 36, 38, 40, 42, and 44.

In certain other examples, the at least one favorable allele of one or more marker loci is selected from the group consisting of S13251-1-K1 allele T; position 1338884 allele T; S07191-1-Q2 allele G; position 1445721 allele G; S14256-1-K1 allele A; position 1464172 allele A; S07192-1-Q20 allele T; position 1470496 allele T; S14242-1-K1 allele A; position 1506154 allele A; S13261-1-K1 allele G; position 1555737 allele G; S14255-1-K1 allele C; position 1567278 allele C; S14251-1-K1 allele C; and position 1593206 allele C. In certain particular examples, the SNP haplotype comprises the marker alleles S13251-1-K1 allele T; S07191-1-Q2 allele G; S14256-1-K1 allele A; S07192-1-Q20 allele T; S14242-1-K1 allele A; S13261-1-K1 allele G; S14255-1-K1 allele C; and S14251-1-K1 allele C. In other particular examples, the SNP haplotype comprises the marker alleles S07191-1-Q2 allele G; S14256-1-K1 allele A; and S07192-1-Q20 allele T.

In certain examples, the detecting comprises amplifying the marker locus or a portion of the marker locus and detecting the resulting amplified marker amplicon. In particular examples, the amplifying comprises: 1) admixing an amplification primer or amplification primer pair and, optionally at least one nucleic acid probe, with a nucleic acid isolated from the first soybean plant or germplasm, wherein the primer or primer pair and optional probe is complementary or partially complementary to at least a portion of the marker locus and is capable of initiating DNA polymerization by a DNA polymerase using the soybean nucleic acid as a template; and 2) extending the primer or primer pair in a DNA polymerization reaction comprising a DNA polymerase and a template nucleic acid to generate at least one amplicon. In particular examples, the detection comprises real time PCR analysis.

In still further aspects, the information disclosed herein regarding marker alleles and SNP haplotypes can be used to aid in the selection of breeding plants, lines, and populations containing tolerance to root-knot nematode for use in introgression of this trait into elite soybean germplasm, or germplasm of proven genetic superiority suitable for variety release. Also provided is a method for introgressing a soybean QTL, marker, or haplotype associated with root-knot nematode tolerance into non-tolerant or less tolerant soybean germplasm. According to the method, markers and/or haplotypes are used to select soybean plants containing the improved tolerance trait. Plants so selected can be used in a soybean breeding program. Through the process of introgression, the QTL, marker, or haplotype associated with improved root-knot nematode tolerance is introduced from plants identified using marker-assisted selection (MAS) to other plants. According to the method, agronomically desirable plants and seeds can be produced containing the QTL, marker, or haplotype associated with root-knot nematode tolerance from germplasm containing the QTL, marker, or haplotype. Sources of improved tolerance are disclosed below.

Also provided herein is a method for producing a soybean plant adapted for conferring improved root-knot nematode tolerance. First, donor soybean plants for a parental line containing the tolerance QTL, marker, and/or haplotype are selected. According to the method, selection can be accomplished via MAS as explained herein. Selected plant material may represent, among others, an inbred line, a hybrid line, a heterogeneous population of soybean plants, or an individual plant. According to techniques well known in the art of plant breeding, this donor parental line is crossed with a second parental line. In some examples, the second parental line is a high yielding line. This cross produces a segregating plant population composed of genetically heterogeneous plants. Plants of the segregating plant population are screened for the tolerance QTL, marker, or haplotype. Further breeding may include, among other techniques, additional crosses with other lines, hybrids, backcrossing, or self-crossing. The result is a line of soybean plants that has improved tolerance to root-knot nematode and optionally also has other desirable traits from one or more other soybean lines.

Soybean plants, seeds, tissue cultures, variants and mutants having improved root-knot nematode tolerance produced by the foregoing methods are also provided. Soybean plants, seeds, tissue cultures, variants and mutants comprising one or more of the marker loci, one or more of the favorable alleles, and/or one or more of the haplotypes and having improved root-knot nematode tolerance are provided. Also provided are isolated nucleic acids, kits, and systems useful for the identification and/or selection methods disclosed herein.

It is to be understood that this invention is not limited to particular embodiments, which can, of course, vary. It is also to be understood that the terminology used herein is for the purpose of describing particular embodiments only, and is not intended to be limiting. Further, all publications referred to herein are incorporated by reference herein for the purpose cited to the same extent as if each was specifically and individually indicated to be incorporated by reference herein.

### DEFINITIONS

As used in this specification and the appended claims, terms in the singular and the singular forms “a,” “an,” and “the,” for example, include plural referents unless the content clearly dictates otherwise. Thus, for example, reference to “plant,” “the plant,” or “a plant” also includes a plurality of plants; also, depending on the context, use of the term “plant” can also include genetically similar or identical progeny of that plant; use of the term “a nucleic acid” optionally includes, as a practical matter, many copies of that nucleic acid molecule; similarly, the term “probe” optionally (and typically) encompasses many similar or identical probe molecules.

Additionally, as used herein, “comprising” is to be interpreted as specifying the presence of the stated features, integers, steps, or components as referred to, but does not preclude the presence or addition of one or more features, integers, steps, or components, or groups thereof. Thus, for example, a kit comprising one pair of oligonucleotide primers may have two or more pairs of oligonucleotide primers. Additionally, the term “comprising” is intended to include examples encompassed by the terms “consisting essentially of” and “consisting of.” Similarly, the term “consisting essentially of” is intended to include examples encompassed by the term “consisting of.”

Certain definitions used in the specification and claims are provided below. In order to provide a clear and consistent understanding of the specification and claims, including the scope to be given such terms, the following definitions are provided:

“Allele” means any of one or more alternative forms of a genetic sequence. In a diploid cell or organism, the two alleles of a given sequence typically occupy corresponding loci on a pair of homologous chromosomes. With regard to a SNP marker, allele refers to the specific nucleotide base present at that SNP locus in that individual plant.

The term “amplifying” in the context of nucleic acid amplification is any process whereby additional copies of a selected nucleic acid (or a transcribed form thereof) are produced. An “amplicon” is an amplified nucleic acid, e.g., a nucleic acid that is produced by amplifying a template nucleic acid by any available amplification method.

“Backcrossing” is a process in which a breeder crosses a progeny variety back to one of the parental genotypes one or more times.

The term “chromosome segment” designates a contiguous linear span of genomic DNA that resides in planta on a single chromosome. “Chromosome interval” refers to a chromosome segment defined by specific flanking marker loci.

“Cultivar” and “variety” are used synonymously and mean a group of plants within a species (e.g., Glycine max) that share certain genetic traits that separate them from other possible varieties within that species. Soybean cultivars are inbred lines produced after several generations of self-pollinations. Individuals within a soybean cultivar are homogeneous, nearly genetically identical, with most loci in the homozygous state.

An “elite line” is an agronomically superior line that has resulted from many cycles of breeding and selection for superior agronomic performance. Numerous elite lines are available and known to those of skill in the art of soybean breeding.

An “elite population” is an assortment of elite individuals or lines that can be used to represent the state of the art in terms of agronomically superior genotypes of a given crop species, such as soybean.

An “exotic soybean strain” or an “exotic soybean germplasm” is a strain or germplasm derived from a soybean not belonging to an available elite soybean line or strain of germplasm. In the context of a cross between two soybean plants or strains of germplasm, an exotic germplasm is not closely related by descent to the elite germplasm with which it is crossed. Most commonly, the exotic germplasm is not derived from any known elite line of soybean, but rather is selected to introduce novel genetic elements (typically novel alleles) into a breeding program.

A “genetic map” is a description of genetic linkage relationships among loci on one or more chromosomes (or linkage groups) within a given species, generally depicted in a diagrammatic or tabular form.

“Genotype” refers to the genetic constitution of a cell or organism.

“Germplasm” means the genetic material that comprises the physical foundation of the hereditary qualities of an organism. As used herein, germplasm includes seeds and living tissue from which new plants may be grown; or, another plant part, such as leaf, stem, pollen, or cells, that may be cultured into a whole plant. Germplasm resources provide sources of genetic traits used by plant breeders to improve commercial cultivars.

An individual is “homozygous” if the individual has only one type of allele at a given locus (e.g., a diploid individual has a copy of the same allele at a locus for each of two homologous chromosomes). An individual is “heterozygous” if more than one allele type is present at a given locus (e.g., a diploid individual with one copy each of two different alleles). The term “homogeneity” indicates that members of a group have the same genotype at one or more specific loci. In contrast, the term “heterogeneity” is used to indicate that individuals within the group differ in genotype at one or more specific loci.

“Introgression” means the entry or introduction of a gene, QTL, marker, haplotype, marker profile, trait, or trait locus from the genome of one plant into the genome of another plant.

The terms “label” and “detectable label” refer to a molecule capable of detection. A detectable label can also include a combination of a reporter and a quencher, such as are employed in FRET probes or TaqMan™ probes. The term “reporter” refers to a substance or a portion thereof that is capable of exhibiting a detectable signal, which signal can be suppressed by a quencher. The detectable signal of the reporter is, e.g., fluorescence in the detectable range. The term “quencher” refers to a substance or portion thereof that is capable of suppressing, reducing, inhibiting, etc., the detectable signal produced by the reporter. As used herein, the terms “quenching” and “fluorescence energy transfer” refer to the process whereby, when a reporter and a quencher are in close proximity, and the reporter is excited by an energy source, a substantial portion of the energy of the excited state nonradiatively transfers to the quencher where it either dissipates nonradiatively or is emitted at a different emission wavelength than that of the reporter.

A “line” or “strain” is a group of individuals of identical parentage that are generally inbred to some degree and that are generally homozygous and homogeneous at most loci (isogenic or near isogenic). A “subline” refers to an inbred subset of descendents that are genetically distinct from other similarly inbred subsets descended from the same progenitor. Traditionally, a subline has been derived by inbreeding the seed from an individual soybean plant selected at the F3 to F5 generation until the residual segregating loci are “fixed” or homozygous across most or all loci. Commercial soybean varieties (or lines) are typically produced by aggregating (“bulking”) the self-pollinated progeny of a single F3 to F5 plant from a controlled cross between two genetically different parents. While the variety typically appears uniform, the self-pollinating variety derived from the selected plant eventually (e.g., F8) becomes a mixture of homozygous plants that can vary in genotype at any locus that was heterozygous in the originally selected F3 to F5 plant. Marker-based sublines that differ from each other based on qualitative polymorphism at the DNA level at one or more specific marker loci are derived by genotyping a sample of seed derived from individual self-pollinated progeny derived from a selected F3-F5 plant. The seed sample can be genotyped directly as seed, or as plant tissue grown from such a seed sample. Optionally, seed sharing a common genotype at the specified locus (or loci) are bulked providing a subline that is genetically homogenous at identified loci important for a trait of interest (e.g., yield, tolerance, etc.).

“Linkage” refers to a phenomenon wherein alleles on the same chromosome tend to segregate together more often than expected by chance if their transmission was independent. Genetic recombination occurs with an assumed random frequency over the entire genome. Genetic maps are constructed by measuring the frequency of recombination between pairs of traits or markers. The closer the traits or markers are to each other on the chromosome, the lower the frequency of recombination, and the greater the degree of linkage. Traits or markers are considered herein to be linked if they generally co-segregate. A 1/100 probability of recombination per generation is defined as a map distance of 1.0 centiMorgan (1.0 cM).

The genetic elements or genes located on a single chromosome segment are physically linked. In some embodiments, the two loci are located in close proximity such that recombination between homologous chromosome pairs does not occur between the two loci during meiosis with high frequency, e.g., such that linked loci co-segregate at least about 90% of the time, e.g., 91%, 92%, 93%, 94%, 95%, 96%, 97%, 98%, 99%, 99.5%, 99.75%, or more of the time. The genetic elements located within a chromosome segment are also genetically linked, typically within a genetic recombination distance of less than or equal to 50 centimorgans (cM), e.g., about 49, 40, 30, 20, 10, 9, 8, 7, 6, 5, 4, 3, 2, 1, 0.75, 0.5, or 0.25 cM or less. That is, two genetic elements within a single chromosome segment undergo recombination during meiosis with each other at a frequency of less than or equal to about 50%, e.g., about 49%, 40%, 30%, 20%, 10%, 9%, 8%, 7%, 6%, 5%, 4%, 3%, 2%, 1%, 0.75%, 0.5%, or 0.25% or less. Closely linked markers display a cross over frequency with a given marker of about 10% or less (the given marker is within about 10 cM of a closely linked marker). Put another way, closely linked loci co-segregate at least about 90% of the time.

With regard to physical position on a chromosome, closely linked markers can be separated, for example, by about 1 megabase (Mb; 1 million nucleotides), about 500 kilobases (Kb; 1000 nucleotides), about 400 Kb, about 300 Kb, about 200 Kb, about 100 Kb, about 50 Kb, about 25 Kb, about 10 Kb, about 5 Kb, about 4 Kb, about 3 Kb, about 2 Kb, about 1 Kb, about 500 nucleotides, about 250 nucleotides, or less.

When referring to the relationship between two genetic elements, such as a genetic element contributing to tolerance and a proximal marker, “coupling” phase linkage indicates the state where the “favorable” allele at the tolerance locus is physically associated on the same chromosome strand as the “favorable” allele of the respective linked marker locus. In coupling phase, both favorable alleles are inherited together by progeny that inherit that chromosome strand. In “repulsion” phase linkage, the “favorable” allele at the locus of interest (e.g., a QTL for tolerance) is physically linked with an “unfavorable” allele at the proximal marker locus, and the two “favorable” alleles are not inherited together (i.e., the two loci are “out of phase” with each other).

“Linkage disequilibrium” refers to a phenomenon wherein alleles tend to remain together in linkage groups when segregating from parents to offspring, with a greater frequency than expected from their individual frequencies.

“Linkage group” refers to traits or markers that generally co-segregate. A linkage group generally corresponds to a chromosomal region containing genetic material that encodes the traits or markers.

“Locus” is a defined segment of DNA.

A “map location,” “map position,” or “relative map position” is an assigned location on a genetic map relative to linked genetic markers where a specified marker can be found within a given species. Map positions are generally provided in centimorgans. A “physical position” or “physical location” is the position, typically in nucleotide bases, of a particular nucleotide, such as a SNP nucleotide, on the chromosome.

“Mapping” is the process of defining the linkage relationships of loci through the use of genetic markers, populations segregating for the markers, and standard genetic principles of recombination frequency.

“Marker” or “molecular marker” is a term used to denote a nucleic acid or amino acid sequence that is sufficiently unique to characterize a specific locus on the genome. Any detectible polymorphic trait can be used as a marker so long as it is inherited differentially and exhibits linkage disequilibrium with a phenotypic trait of interest. A number of soybean markers have been mapped and linkage groups created, as described in Cregan, P. B., et al., “An Integrated Genetic Linkage Map of the Soybean Genome” (1999) Crop Science 39:1464-90, and more recently in Choi, et al., “A Soybean Transcript Map: Gene Distribution, Haplotype and Single-Nucleotide Polymorphism Analysis” (2007) Genetics 176:685-96. Many soybean markers are publicly available at the USDA affiliated soybase website (www.soybase.org). All markers are used to define a specific locus on the soybean genome. Large numbers of these markers have been mapped. Each marker is therefore an indicator of a specific segment of DNA, having a unique nucleotide sequence. The map positions provide a measure of the relative positions of particular markers with respect to one another. When a trait is stated to be linked to a given marker, it will be understood that the actual DNA segment whose sequence affects the trait generally co-segregates with the marker. More precise and definite localization of a trait can be obtained if markers are identified on both sides of the trait. By measuring the appearance of the marker(s) in progeny of crosses, the existence of the trait can be detected by relatively simple molecular tests without actually evaluating the appearance of the trait itself, which can be difficult and time-consuming because the actual evaluation of the trait requires growing plants to a stage and/or under environmental conditions where the trait can be expressed. Molecular markers have been widely used to determine genetic composition in soybeans. “Marker assisted selection” refers to the process of selecting a desired trait or traits in a plant or plants by detecting one or more nucleic acids from the plant, where the nucleic acid is linked to the desired trait, and then selecting the plant or germplasm possessing those one or more nucleic acids.

“Haplotype” refers to a combination of particular alleles present within a particular plant's genome at two or more linked marker loci, for instance at two or more loci on a particular linkage group. For instance, in one example, two specific marker loci on LG-O are used to define a haplotype for a particular plant. In still further examples, 3, 4, 5, 6, 7, 8, 9, 10, 11, 12, 13, 14, 15, 16, 17, 18, 19, 20, or more linked marker loci are used to define a haplotype for a particular plant.

The term “plant” includes reference to an immature or mature whole plant, including a plant from which seed or grain or anthers have been removed. Seed or embryo that will produce the plant is also considered to be the plant.

“Plant parts” means any portion or piece of a plant, including leaves, stems, buds, roots, root tips, anthers, seed, grain, embryo, pollen, ovules, flowers, cotyledons, hypocotyls, pods, flowers, shoots, stalks, tissues, tissue cultures, cells, and the like.

“Polymorphism” means a change or difference between two related nucleic acids. A “nucleotide polymorphism” refers to a nucleotide that is different in one sequence when compared to a related sequence when the two nucleic acids are aligned for maximal correspondence.

“Polynucleotide,” “polynucleotide sequence,” “nucleic acid sequence,” “nucleic acid fragment,” and “oligonucleotide” are used interchangeably herein. These terms encompass nucleotide sequences and the like. A polynucleotide is a polymer of nucleotides that is single- or multi-stranded, that optionally contains synthetic, non-natural, or altered RNA or DNA nucleotide bases. A DNA polynucleotide may be comprised of one or more strands of cDNA, genomic DNA, synthetic DNA, or mixtures thereof.

“Primer” refers to an oligonucleotide (synthetic or occurring naturally), which is capable of acting as a point of initiation of nucleic acid synthesis or replication along a complementary strand when placed under conditions in which synthesis of a complementary strand is catalyzed by a polymerase. Typically, primers are about 10 to 30 nucleotides in length, but longer or shorter sequences can be employed. Primers may be provided in double-stranded form, though the single-stranded form is more typically used. A primer can further contain a detectable label, for example a 5′ end label.

“Probe” refers to an oligonucleotide (synthetic or occurring naturally) that is complementary (though not necessarily fully complementary) to a polynucleotide of interest and forms a duplexed structure by hybridization with at least one strand of the polynucleotide of interest. Typically, probes are oligonucleotides from 10 to 50 nucleotides in length, but longer or shorter sequences can be employed. A probe can further contain a detectable label.

“Quantitative trait loci” or “QTL” refer to the genetic elements controlling a quantitative trait.

“Recombination frequency” is the frequency of a crossing over event (recombination) between two genetic loci. Recombination frequency can be observed by following the segregation of markers and/or traits during meiosis.

“Tolerance and “improved tolerance” are used interchangeably herein and refer to any type of increase in resistance or tolerance to, or any type of decrease in susceptibility to root-knot nematode. A “tolerant plant” or “tolerant plant variety” need not possess absolute or complete tolerance to root-knot nematode. Instead, a “tolerant plant,” “tolerant plant variety,” or a plant or plant variety with “improved tolerance” will have a level of resistance or tolerance to root-knot nematode which is higher than that of a comparable susceptible plant or variety.

“Self crossing” or “self pollination” or “selfing” is a process through which a breeder crosses a plant with itself; for example, a second-generation hybrid F2 with itself to yield progeny designated F2:3.

“SNP” or “single nucleotide polymorphism” means a sequence variation that occurs when a single nucleotide (A, T, C, or G) in the genome sequence is altered or variable. “SNP markers” exist when SNPs are mapped to sites on the soybean genome.

The term “yield” refers to the productivity per unit area of a particular plant product of commercial value. For example, yield of soybean is commonly measured in bushels of seed per acre or metric tons of seed per hectare per season. Yield is affected by both genetic and environmental factors. Yield is the final culmination of all agronomic traits.

Markers and Haplotypes Associated with Root-Knot Nematode Tolerance:

Provided are markers and haplotypes associated with tolerance of soybean plants to root-knot nematode, as well as related primers and/or probes and methods for the use of any of the foregoing for identifying and/or selecting soybean plants with improved tolerance to root-knot nematode. A method for determining the presence or absence of at least one allele of a particular marker or haplotype associated with tolerance to root-knot nematode comprises analyzing genomic DNA from a soybean plant or germplasm to determine if at least one, or a plurality, of such markers is present or absent and if present, determining the allelic form of the marker(s). If a plurality of markers on a single linkage group are investigated, this information regarding the markers present in the particular plant or germplasm can be used to determine a haplotype for that plant/germplasm.

In certain examples, plants or germplasm are identified that have at least one favorable allele, marker, and/or haplotype that positively correlates with tolerance or improved tolerance. However, in other examples, it is useful to identify alleles, markers, and/or haplotypes that negatively correlate with tolerance, for example to eliminate such plants or germplasm from subsequent rounds of breeding.

Any marker associated with a root-knot nematode tolerance QTL is useful. Further, any suitable type of marker can be used, including Restriction Fragment Length Polymorphisms (RFLPs), Single Sequence Repeats (SSRs), Target Region Amplification Polymorphisms (TRAPs), Isozyme Electrophoresis, Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length Polymorphisms (AFLPs), and Single Nucleotide Polymorphisms (SNPs). Additionally, other types of molecular markers known in the art or phenotypic traits may also be used as markers in the methods.

Markers that map closer to a root-knot tolerance QTL are generally preferred over markers that map farther from such a QTL. Marker loci are especially useful when they are closely linked to a root-knot nematode tolerance QTL. Thus, in one example, marker loci display an inter-locus cross-over frequency of about 10% or less, about 9% or less, about 8% or less, about 7% or less, about 6% or less, about 5% or less, about 4% or less, about 3% or less, about 2% or less, about 1% or less, about 0.75% or less, about 0.5% or less, or about 0.25% or less with a root-knot nematode tolerance QTL to which they are linked. Thus, the loci are separated from the QTL to which they are linked by about 10 cM, 9 cM, 8 cM, 7 cM, 6 cM, 5 cM, 4 cM, 3 cM, 2 cM, 1 cM, 0.75 cM, 0.5 cM, or 0.25 cM or less.

In certain examples, multiple marker loci that collectively make up a haplotype are investigated, for instance 2, 3, 4, 5, 6, 7, 8, 9, 10, 11, 12, 13, 14, 15, 16, 17, 18, 19, 20, 21, 22, 23, 24, 25, 26, 27, 28, 29, 30, or more marker loci.

In addition to the markers discussed herein, information regarding useful soybean markers can be found, for example, on the USDA's Soybase website, available at www.soybase.org. One of skill in the art will recognize that the identification of favorable marker alleles may be germplasm-specific. One of skill will also recognize that methods for identifying the favorable alleles are routine and well known in the art, and furthermore, that the identification and use of such favorable alleles is well within the scope of the invention.

The use of marker assisted selection (MAS) to select a soybean plant or germplasm based upon detection of a particular marker or haplotype of interest is provided. For instance, in certain examples, a soybean plant or germplasm possessing a certain predetermined favorable marker allele or haplotype will be selected via MAS. Using MAS, soybean plants or germplasm can be selected for markers or marker alleles that positively correlate with tolerance, without actually raising soybean and measuring for tolerance (or, contrawise, soybean plants can be selected against if they possess markers that negatively correlate with tolerance). MAS is a powerful tool to select for desired phenotypes and for introgressing desired traits into cultivars of soybean (e.g., introgressing desired traits into elite lines). MAS is easily adapted to high throughput molecular analysis methods that can quickly screen large numbers of plant or germplasm genetic material for the markers of interest and is much more cost effective than raising and observing plants for visible traits.

In some examples, molecular markers are detected using a suitable amplification-based detection method. Typical amplification methods include various polymerase based replication methods, including the polymerase chain reaction (PCR), ligase mediated methods, such as the ligase chain reaction (LCR), and RNA polymerase based amplification (e.g., by transcription) methods. In these types of methods, nucleic acid primers are typically hybridized to the conserved regions flanking the polymorphic marker region. In certain methods, nucleic acid probes that bind to the amplified region are also employed. In general, synthetic methods for making oligonucleotides, including primers and probes, are well known in the art. For example, oligonucleotides can be synthesized chemically according to the solid phase phosphoramidite triester method described by Beaucage and Caruthers (1981) Tetrahedron Letts 22:1859-1862, e.g., using a commercially available automated synthesizer, e.g., as described in Needham-VanDevanter, et al. (1984) Nucleic Acids Res. 12:6159-6168. Oligonucleotides, including modified oligonucleotides, can also be ordered from a variety of commercial sources known to persons of skill in the art.

It will be appreciated that suitable primers and probes to be used can be designed using any suitable method. It is not intended that the invention be limited to any particular primer, primer pair, or probe. For example, primers can be designed using any suitable software program, such as LASERGENE® or Primer3.

It is not intended that the primers be limited to generating an amplicon of any particular size. For example, the primers used to amplify the marker loci and alleles herein are not limited to amplifying the entire region of the relevant locus. In some examples, marker amplification produces an amplicon at least 20 nucleotides in length, or alternatively, at least 50 nucleotides in length, or alternatively, at least 100 nucleotides in length, or alternatively, at least 200 nucleotides in length, or alternatively, at least 300 nucleotides in length, or alternatively, at least 400 nucleotides in length, or alternatively, at least 500 nucleotides in length, or alternatively, at least 1000 nucleotides in length, or alternatively, at least 2000 nucleotides in length, or alternatively.

PCR, RT-PCR, and LCR are common amplification and amplification-detection methods for amplifying nucleic acids of interest (e.g., those comprising marker loci), facilitating detection of the markers. Details regarding the use of these and other amplification methods are well known in the art and can be found in any of a variety of standard texts. Details for these techniques can also be found in numerous journal and patent references, such as Mullis, et al. (1987) U.S. Pat. No. 4,683,202; Arnheim & Levinson (1990) C&EN 36-47; Kwoh et al. (1989) Proc. Natl. Acad. Sci. USA 86:1173; Guatelli et al. (1990) Proc. Natl. Acad. Sci. USA87:1874; Lomell et al. (1989) J. Clin. Chem 35:1826; Landegren et al. (1988) Science 241:1077-1080; Van Brunt (1990) Biotechnology 8:291-294; Wu and Wallace (1989) Gene 4:560; Barringer et al. (1990) Gene 89:117; and Sooknanan and Malek (1995) Biotechnology 13:563-564.

Such nucleic acid amplification techniques can be applied to amplify and/or detect nucleic acids of interest, such as nucleic acids comprising marker loci. Amplification primers for amplifying useful marker loci and suitable probes to detect useful marker loci or to genotype alleles, such as SNP alleles, are provided. For example, exemplary primers and probes are provided in Table 4. However, one of skill will immediately recognize that other primer and probe sequences could also be used. For instance, primers to either side of the given primers can be used in place of the given primers, so long as the primers can amplify a region that includes the allele to be detected, as can primers and probes directed to other marker loci. Further, it will be appreciated that the precise probe to be used for detection can vary, e.g., any probe that can identify the region of a marker amplicon to be detected can be substituted for those examples provided herein. Further, the configuration of the amplification primers and detection probes can, of course, vary. Thus, the compositions and methods are not limited to the primers and probes specifically recited herein.

In certain examples, probes will possess a detectable label. Any suitable label can be used with a probe. Detectable labels suitable for use with nucleic acid probes include, for example, any composition detectable by spectroscopic, radioisotopic, photochemical, biochemical, immunochemical, electrical, optical, or chemical means. Useful labels include biotin for staining with labeled streptavidin conjugate, magnetic beads, fluorescent dyes, radiolabels, enzymes, and colorimetric labels. Other labels include ligands, which bind to antibodies labeled with fluorophores, chemiluminescent agents, and enzymes. A probe can also constitute radiolabelled PCR primers that are used to generate a radiolabelled amplicon. Labeling strategies for labeling nucleic acids and corresponding detection strategies can be found, e.g., in Haugland (1996) Handbook of Fluorescent Probes and Research Chemicals Sixth Edition by Molecular Probes, Inc. (Eugene, Oreg.); or Haugland (2001) Handbook of Fluorescent Probes and Research Chemicals Eighth Edition by Molecular Probes, Inc. (Eugene, Oreg.).

Detectable labels may also include reporter-quencher pairs, such as are employed in Molecular Beacon and TaqMan™ probes. The reporter may be a fluorescent organic dye modified with a suitable linking group for attachment to the oligonucleotide, such as to the terminal 3′ carbon or terminal 5′ carbon. The quencher may also be an organic dye, which may or may not be fluorescent. Generally, whether the quencher is fluorescent or simply releases the transferred energy from the reporter by non-radiative decay, the absorption band of the quencher should at least substantially overlap the fluorescent emission band of the reporter to optimize the quenching. Non-fluorescent quenchers or dark quenchers typically function by absorbing energy from excited reporters, but do not release the energy radiatively.

Selection of appropriate reporter-quencher pairs for particular probes may be undertaken in accordance with known techniques. Fluorescent and dark quenchers and their relevant optical properties from which exemplary reporter-quencher pairs may be selected are listed and described, for example, in Berlman, Handbook of Fluorescence Spectra of Aromatic Molecules, 2nd ed., Academic Press, New York, 1971, the content of which is incorporated herein by reference. Examples of modifying reporters and quenchers for covalent attachment via common reactive groups that can be added to an oligonucleotide in the present invention may be found, for example, in Haugland (2001) Handbook of Fluorescent Probes and Research Chemicals Eighth Edition by Molecular Probes, Inc. (Eugene, Oreg.), the content of which is incorporated herein by reference.

In certain examples, reporter-quencher pairs are selected from xanthene dyes including fluorescein and rhodamine dyes. Many suitable forms of these compounds are available commercially with substituents on the phenyl groups, which can be used as the site for bonding or as the bonding functionality for attachment to an oligonucleotide. Another useful group of fluorescent compounds for use as reporters is the naphthylamines, having an amino group in the alpha or beta position. Included among such naphthylamino compounds are 1-dimethylaminonaphthyl-5 sulfonate, 1-anilino-8-naphthalene sulfonate and 2-p-touidinyl-6-naphthalene sulfonate. Other dyes include 3-phenyl-7-isocyanatocoumarin; acridines such as 9-isothiocyanatoacridine; N-(p-(2-benzoxazolyl)phenyl)maleimide; benzoxadiazoles; stilbenes; pyrenes and the like. In certain other examples, the reporters and quenchers are selected from fluorescein and rhodamine dyes. These dyes and appropriate linking methodologies for attachment to oligonucleotides are well known in the art.

Suitable examples of reporters may be selected from dyes such as SYBR green, 5-carboxyfluorescein (5-FAMT™ available from Applied Biosystems of Foster City, Calif.), 6-carboxyfluorescein (6-FAM), tetrachloro-6-carboxyfluorescein (TET), 2,7-dimethoxy-4,5-dichloro-6-carboxyfluorescein, hexachloro-6-carboxyfluorescein (HEX), 6-carboxy-2′,4,7,7′-tetrachlorofluorescein (6-TET™ available from Applied Biosystems), carboxy-X-rhodamine (ROX), 6-carboxy-4′,5′-dichloro-2′,7′-dimethoxyfluorescein (6-JOE™ available from Applied Biosystems), VIC™ dye products available from Molecular Probes, Inc., NED™ dye products available from available from Applied Biosystems, and the like. Suitable examples of quenchers may be selected from 6-carboxy-tetramethyl-rhodamine, 4-(4-dimethylaminophenylazo) benzoic acid (DABYL), tetramethylrhodamine (TAMRA), BHQ-0™, BHQ-1™, BHQ-2™, and BHQ-3™, each of which are available from Biosearch Technologies, Inc. of Novato, Calif., QSY-7™, QSY9™, QSY-21™ and QSY-35™, each of which are available from Molecular Probes, Inc., and the like.

In one aspect, real time PCR or LCR is performed on the amplification mixtures described herein, e.g., using molecular beacons or TaqMan™ probes. A molecular beacon (MB) is an oligonucleotide that, under appropriate hybridization conditions, self-hybridizes to form a stem and loop structure. The MB has a label and a quencher at the termini of the oligonucleotide; thus, under conditions that permit intra-molecular hybridization, the label is typically quenched (or at least altered in its fluorescence) by the quencher. Under conditions where the MB does not display intra-molecular hybridization (e.g., when bound to a target nucleic acid, such as to a region of an amplicon during amplification), the MB label is unquenched. Details regarding standard methods of making and using MBs are well established in the literature and MBs are available from a number of commercial reagent sources. See also, e.g., Leone, et al., (1995) Nucleic Acids Res. 26:2150-2155; Tyagi and Kramer (1996) Nature Biotechnology 14:303-308; Blok and Kramer (1997) Mol Cell Probes 11:187-194; Hsuih et al. (1997) J Clin Microbiol 34:501-507; Kostrikis et al. (1998) Science 279:1228-1229; Sokol et al. (1998) Proc. Natl. Acad. Sci. U.S.A. 95:11538-11543; Tyagi et al. (1998) Nature Biotechnology 16:49-53; Bonnet et al. (1999) Proc. Natl. Acad. Sci. U.S.A. 96:6171-6176; Fang et al. (1999) J. Am. Chem. Soc. 121:2921-2922; Marras et al. (1999) Genet. Anal. Biomol. Eng. 14:151-156; and, Vet et al. (1999) Proc. Natl. Acad. Sci. U.S.A. 96:6394-6399. Additional details regarding MB construction and use are also found in the patent literature, e.g., U.S. Pat. Nos. 5,925,517; 6,150,097; and 6,037,130.

Another real-time detection method is the 5′-exonuclease detection method, also called the TaqMan™ assay, as set forth in U.S. Pat. Nos. 5,804,375; 5,538,848; 5,487,972; and 5,210,015, each of which is hereby incorporated by reference in its entirety. In the TaqMan™ assay, a modified probe, typically 10-30 nucleotides in length, is employed during PCR which binds intermediate to or between the two members of the amplification primer pair. The modified probe possesses a reporter and a quencher and is designed to generate a detectable signal to indicate that it has hybridized with the target nucleic acid sequence during PCR. As long as both the reporter and the quencher are on the probe, the quencher stops the reporter from emitting a detectable signal. However, as the polymerase extends the primer during amplification, the intrinsic 5′ to 3′ nuclease activity of the polymerase degrades the probe, separating the reporter from the quencher, and enabling the detectable signal to be emitted. Generally, the amount of detectable signal generated during the amplification cycle is proportional to the amount of product generated in each cycle.

It is well known that the efficiency of quenching is a strong function of the proximity of the reporter and the quencher, i.e., as the two molecules get closer, the quenching efficiency increases. As quenching is strongly dependent on the physical proximity of the reporter and quencher, the reporter and the quencher are typically attached to the probe within a few nucleotides of one another, usually within 30 nucleotides of one another, or within 6 to 16 nucleotides. Typically, this separation is achieved by attaching one member of a reporter-quencher pair to the 5′ end of the probe and the other member to a nucleotide about 6 to 16 nucleotides away, in some cases at the 3′ end of the probe.

Separate detection probes can also be omitted in amplification/detection methods, e.g., by performing a real time amplification reaction that detects product formation by modification of the relevant amplification primer upon incorporation into a product, incorporation of labeled nucleotides into an amplicon, or by monitoring changes in molecular rotation properties of amplicons as compared to unamplified precursors (e.g., by fluorescence polarization).

One example of a suitable real-time detection technique that does not use a separate probe that binds intermediate to the two primers is the KASPar detection system/method, which is well known in the art. In KASPar, two allele specific primers are designed such that the 3′ nucleotide of each primer hybridizes to the polymorphic base. For example, if the SNP is an A/C polymorphism, one of the primers would have an “A” in the 3′ position, while the other primer would have a “C” in the 3′ position. Each of these two allele specific primers also has a unique tail sequence on the 5′ end of the primer. A common reverse primer is employed that amplifies in conjunction with either of the two allele specific primers. Two 5′ fluor-labeled reporter oligos are also included in the reaction mix, one designed to interact with each of the unique tail sequences of the allele-specific primers. Lastly, one quencher oligo is included for each of the two reporter oligos, the quencher oligo being complementary to the reporter oligo and being able to quench the fluor signal when bound to the reporter oligo. During PCR, the allele-specific primers and reverse primers bind to complementary DNA, allowing amplification of the amplicon to take place. During a subsequent cycle, a complementary nucleic acid strand containing a sequence complementary to the unique tail sequence of the allele-specific primer is created. In a further cycle, the reporter oligo interacts with this complementary tail sequence, acting as a labeled primer. Thus, the product created from this cycle of PCR is a fluorescently-labeled nucleic acid strand. Because the label incorporated into this amplification product is specific to the allele specific primer that resulted in the amplification, detecting the specific fluor presenting a signal can be used to determine the SNP allele that was present in the sample.

Further, it will be appreciated that amplification is not a requirement for marker detection for example, one can directly detect unamplified genomic DNA simply by performing a Southern blot on a sample of genomic DNA. Procedures for performing Southern blotting, amplification e.g., (PCR, LCR, or the like), and many other nucleic acid detection methods are well established and are taught, e.g., in Sambrook et al. Molecular Cloning—A Laboratory Manual (3d ed.) Vol. 1-3, Cold Spring Harbor Laboratory, Cold Spring Harbor, N.Y., 2000 (“Sambrook”); Current Protocols in Molecular Biology, F. M. Ausubel et al., eds., Current Protocols, a joint venture between Greene Publishing Associates, Inc. and John Wiley & Sons, Inc., (supplemented through 2002) (“Ausubel”); and, PCR Protocols A Guide to Methods and Applications (Innis et al., eds) Academic Press Inc. San Diego, Calif. (1990) (“Innis”). Additional details regarding detection of nucleic acids in plants can also be found, e.g., in Plant Molecular Biology (1993) Croy (ed.) BIOS Scientific Publishers, Inc.

Other techniques for detecting SNPs can also be employed, such as allele specific hybridization (ASH) or nucleic acid sequencing techniques. ASH technology is based on the stable annealing of a short, single-stranded, oligonucleotide probe to a completely complementary single-stranded target nucleic acid. Detection is via an isotopic or non-isotopic label attached to the probe. For each polymorphism, two or more different ASH probes are designed to have identical DNA sequences except at the polymorphic nucleotides. Each probe will have exact homology with one allele sequence so that the range of probes can distinguish all the known alternative allele sequences. Each probe is hybridized to the target DNA. With appropriate probe design and hybridization conditions, a single-base mismatch between the probe and target DNA will prevent hybridization.

Real-time amplification assays, including MB or TaqMan™ based assays, are especially useful for detecting SNP alleles. In such cases, probes are typically designed to bind to the amplicon region that includes the SNP locus, with one allele-specific probe being designed for each possible SNP allele. For instance, if there are two known SNP alleles for a particular SNP locus, “A” or “C,” then one probe is designed with an “A” at the SNP position, while a separate probe is designed with a “C” at the SNP position. While the probes are typically identical to one another other than at the SNP position, they need not be. For instance, the two allele-specific probes could be shifted upstream or downstream relative to one another by one or more bases. However, if the probes are not otherwise identical, they should be designed such that they bind with approximately equal efficiencies, which can be accomplished by designing under a strict set of parameters that restrict the chemical properties of the probes. Further, a different detectable label, for instance a different reporter-quencher pair, is typically employed on each different allele-specific probe to permit differential detection of each probe. In certain examples, each allele-specific probe for a certain SNP locus is 13-18 nucleotides in length, dual-labeled with a florescence quencher at the 3′ end and either the 6-FAM (6-carboxyfluorescein) or VIC (4,7,2′-trichloro-7′-phenyl-6-carboxyfluorescein) fluorophore at the 5′ end.

To effectuate SNP allele detection, a real-time PCR reaction can be performed using primers that amplify the region including the SNP locus, the reaction being performed in the presence of all allele-specific probes for the given SNP locus. By then detecting signal for each detectable label employed and determining which detectable label(s) demonstrated an increased signal, a determination can be made of which allele-specific probe(s) bound to the amplicon and, thus, which SNP allele(s) the amplicon possessed. For instance, when 6-FAM- and VIC-labeled probes are employed, the distinct emission wavelengths of 6-FAM (518 nm) and VIC (554 nm) can be captured. A sample that is homozygous for one allele will have fluorescence from only the respective 6-FAM or VIC fluorophore, while a sample that is heterozygous at the analyzed locus will have both 6-FAM and VIC fluorescence.

Introgression of root-knot nematode tolerance into less tolerant soybean germplasm is provided. Any method for introgressing a QTL or marker into soybean plants known to one of skill in the art can be used. Typically, a first soybean germplasm that contains tolerance to root-knot nematode derived from a particular marker or haplotype and a second soybean germplasm that lacks such tolerance derived from the marker or haplotype are provided. The first soybean germplasm may be crossed with the second soybean germplasm to provide progeny soybean germplasm. These progeny germplasm are screened to determine the presence of root-knot nematode tolerance derived from the marker or haplotype, and progeny that tests positive for the presence of tolerance derived from the marker or haplotype are selected as being soybean germplasm into which the marker or haplotype has been introgressed. Methods for performing such screening are well known in the art and any suitable method can be used.

One application of MAS is to use the tolerance markers or haplotypes to increase the efficiency of an introgression or backcrossing effort aimed at introducing a tolerance trait into a desired (typically high yielding) background. In marker assisted backcrossing of specific markers from a donor source, e.g., to an elite genetic background, one selects among backcross progeny for the donor trait and then uses repeated backcrossing to the elite line to reconstitute as much of the elite background's genome as possible.

Thus, the markers and methods can be utilized to guide marker assisted selection or breeding of soybean varieties with the desired complement (set) of allelic forms of chromosome segments associated with superior agronomic performance (tolerance, along with any other available markers for yield, disease tolerance, etc.). Any of the disclosed marker alleles or haplotypes can be introduced into a soybean line via introgression, by traditional breeding (or introduced via transformation, or both) to yield a soybean plant with superior agronomic performance. The number of alleles associated with tolerance that can be introduced or be present in a soybean plant ranges from 1 to the number of alleles disclosed herein, each integer of which is incorporated herein as if explicitly recited.

This also provides a method of making a progeny soybean plant and these progeny soybean plants, per se. The method comprises crossing a first parent soybean plant with a second soybean plant and growing the female soybean plant under plant growth conditions to yield soybean plant progeny. Methods of crossing and growing soybean plants are well within the ability of those of ordinary skill in the art. Such soybean plant progeny can be assayed for alleles associated with tolerance and, thereby, the desired progeny selected. Such progeny plants or seed can be sold commercially for soybean production, used for food, processed to obtain a desired constituent of the soybean, or further utilized in subsequent rounds of breeding. At least one of the first or second soybean plants is a soybean plant that comprises at least one of the markers or haplotypes associated with tolerance, such that the progeny are capable of inheriting the marker or haplotype.

Often, a method is applied to at least one related soybean plant such as from progenitor or descendant lines in the subject soybean plants pedigree such that inheritance of the desired tolerance can be traced. The number of generations separating the soybean plants being subject to the methods will generally be from 1 to 20, commonly 1 to 5, and typically 1, 2, or 3 generations of separation, and quite often a direct descendant or parent of the soybean plant will be subject to the method (i.e., 1 generation of separation).

Genetic diversity is important for long-term genetic gain in any breeding program. With limited diversity, genetic gain will eventually plateau when all of the favorable alleles have been fixed within the elite population. One objective is to incorporate diversity into an elite pool without losing the genetic gain that has already been made and with the minimum possible investment. MAS provides an indication of which genomic regions and which favorable alleles from the original ancestors have been selected for and conserved over time, facilitating efforts to incorporate favorable variation from exotic germplasm sources (parents that are unrelated to the elite gene pool) in the hopes of finding favorable alleles that do not currently exist in the elite gene pool.

For example, the markers, haplotypes, primers, and probes can be used for MAS involving crosses of elite lines to exotic soybean lines (elite X exotic) by subjecting the segregating progeny to MAS to maintain major yield alleles, along with the tolerance marker alleles herein.

As an alternative to standard breeding methods of introducing traits of interest into soybean (e.g., introgression), transgenic approaches can also be used to create transgenic plants with the desired traits. In these methods, exogenous nucleic acids that encode a desired QTL, marker, or haplotype are introduced into target plants or germplasm. For example, a nucleic acid that codes for a root-knot nematode tolerance trait is cloned, e.g., via positional cloning, and introduced into a target plant or germplasm.

Experienced plant breeders can recognize root-knot nematode tolerant soybean plants in the field, and can select the tolerant individuals or populations for breeding purposes or for propagation. In this context, the plant breeder recognizes “tolerant” and “non-tolerant” or “susceptible” soybean plants. However, plant tolerance is a phenotypic spectrum consisting of extremes in tolerance and susceptibility, as well as a continuum of intermediate tolerance phenotypes. Evaluation of these intermediate phenotypes using reproducible assays are of value to scientists who seek to identify genetic loci that impart tolerance, to conduct marker assisted selection for tolerant populations, and to use introgression techniques to breed a tolerance trait into an elite soybean line, for example.

To that end, screening and selection of tolerant soybean plants may be performed, for example, by exposing plants to root-knot nematode in a greenhouse assay and selecting those plants showing tolerance to the nematodes. Screening may also be done in research and/or crop fields using known methods to assess the presence and type of root-knot nematode, the level of infestation, and the plant phenotype. Any such assay known to the art may be used, e.g., as described in Hussey and Boerma (1981) Crop Sci. 21:794-796; Luzzi, et al. (1994) Crop Sci. 34:1240-1243; and Allen, et al. (2005) Plant Health Progress doi:10.1094/PHP-2005-0606-01.RS, each of which is incorporated herein by reference in its entirety, and/or as described in the Examples hereof.

In some examples, a kit or an automated system for detecting markers or haplotypes, and/or for correlating the markers or haplotypes with a desired phenotype (e.g., root-knot nematode tolerance), are provided. Thus, a typical kit can include a set of marker probes and/or primers configured to detect at least one favorable allele of one or more marker locus associated with tolerance, improved tolerance, or susceptibility to root-knot nematode. These probes or primers can be configured, for example, to detect the marker alleles noted in the tables and examples herein, e.g., using any available allele detection format, such as solid or liquid phase array based detection, microfluidic-based sample detection, etc. The kits can further include packaging materials for packaging the probes, primers, or instructions; controls, such as control amplification reactions that include probes, primers, and/or template nucleic acids for amplifications; molecular size markers; or the like.

A typical system can also include a detector that is configured to detect one or more signal outputs from the set of marker probes or primers, or amplicon thereof, thereby identifying the presence or absence of the allele. A wide variety of signal detection apparatus are available, including photo multiplier tubes, spectrophotometers, CCD arrays, scanning detectors, phototubes and photodiodes, microscope stations, galvo-scans, microfluidic nucleic acid amplification detection appliances, and the like. The precise configuration of the detector will depend, in part, on the type of label used to detect the marker allele, as well as the instrumentation that is most conveniently obtained for the user. Detectors that detect fluorescence, phosphorescence, radioactivity, pH, charge, absorbance, luminescence, temperature, magnetism or the like can be used. Typical detector examples include light (e.g., fluorescence) detectors or radioactivity detectors. For example, detection of a light emission (e.g., a fluorescence emission) or other probe label is indicative of the presence or absence of a marker allele. Fluorescent detection is generally used for detection of amplified nucleic acids (however, upstream and/or downstream operations can also be performed on amplicons, which can involve other detection methods). In general, the detector detects one or more label (e.g., light) emission from a probe label, which is indicative of the presence or absence of a marker allele. The detector(s) optionally monitors one or a plurality of signals from an amplification reaction. For example, the detector can monitor optical signals that correspond to “real time” amplification assay results.

System or kit instructions that describe how to use the system or kit and/or that correlate the presence or absence of the allele with the predicted tolerance or susceptibility phenotype are also provided. For example, the instructions can include at least one look-up table that includes a correlation between the presence or absence of the favorable allele(s) and the predicted tolerance or improved tolerance. The precise form of the instructions can vary depending on the components of the system, e.g., they can be present as system software in one or more integrated unit of the system (e.g., a microprocessor, computer or computer readable medium), or can be present in one or more units (e.g., computers or computer readable media) operably coupled to the detector.

Isolated nucleic acids comprising a nucleic acid sequence coding for tolerance or susceptibility to root-knot nematode, or capable of detecting such a phenotypic trait, or sequences complementary thereto, are also included. In certain examples, the isolated nucleic acids are capable of hybridizing under stringent conditions to nucleic acids of a soybean cultivar displaying tolerance to root-knot nematode, for instance to particular markers, including one or more of S13251-1-K1, S07191-1-Q2, S14256-1-K1, S07192-1-Q20, S14242-1-K1, S13261-1-K1, S14255-1-K1, and S14251-1-K1. Vectors comprising such nucleic acids, expression products of such vectors expressed in a host compatible therewith, antibodies to the expression product (both polyclonal and monoclonal), and antisense nucleic acids are also included.

As the parental line having root-knot nematode tolerance, any line known to the art or disclosed herein may be used. Also included are soybean plants produced by any of the foregoing methods. Seed of a soybean germplasm produced by crossing a soybean variety having a marker or haplotype associated with root-knot nematode tolerance with a soybean variety lacking such marker or haplotype, and progeny thereof, is also included.

The present invention is illustrated by the following examples. The foregoing and following description of the present invention and the various examples are not intended to be limiting of the invention but rather are illustrative thereof. Hence, it will be understood that the invention is not limited to the specific details of these examples.

### EXAMPLES

### Example 1

### Phenotyping a Mapping Population

A mapping population comprising 107 individual plants from three F3 mapping populations derived by crossing the root-knot nematode tolerant line 95Y20 with root-knot nematode susceptible lines 94Y70 (55 F3 individuals), 94Y90 (47 individuals), and 94Y80 (5 individuals) was obtained. The plants in the population were phenotyped for resistance to root-knot nematode as follows: Plants were evaluated and phenotypically scored for RKI tolerance in a greenhouse screeing protocol essentially as described by Hussey and Boerma (1981) Crop Sci. 21:794-796. Five reps are planted with at least 3 seeds each and evaluated for visual gall score along with susceptible check varieties such as Pickett or Bossier, and tolerant checks such as PI96354, PI417444, Forrest, or Bragg (varieties identified by Luzzi, et al. (1994) Crop Sci 34:1240-1243; and Allen, et al. (2005) Plant Health Progress doi:10.1094/PHP-2005-0606-01.RS). RKI scoring was done when root gall formations were adequately developed on the susceptible check, typically about 45-60 after the initial inoculation of 2000-4000 eggs/J2 per plant. Soybean gall formation was measured by visually counting the number of galls on the entire root. This count was translated into the 0-5 scoring system reported in Hussey and Boerma (1981) and shown in Table 1:

TABLE 1
Hussey and Boerma (1981) scoring system

| Gall Score | # Galls |
|---|---|
| 0 | 0 |
| 1 | 1-2 |
| 2 | 3-10 |
| 3 | 11-30 |
| 4 | 31-100 |
| 5 | >100 |

These scores are then converted to a 1-9 score, wherein 1 is very susceptible to RKI (high gall count) and 9 is very resistant (no or low gall count), as shown in Table 2:

TABLE 2
Root-knot nematode resistance 1-9 scoring system

| | Conversion to |
|---|---|
| Gall Score | 1-9 scale |
| 0.0-1.0 | 9 |
| 1.1-1.5 | 8 |
| 1.6-2.0 | 7 |
| 2.1-2.5 | 6 |
| 2.6-3.0 | 5 |
| 3.1-3.5 | 4 |
| 3.6-4.0 | 3 |
| 4.1-4.5 | 2 |
| 4.6-5.0 | 1 |

The results of this phenotypic analysis for the plants from the mapping population are set forth in Table 3.

TABLE 3
Phenotypic scoring of mapping population

| | | | Percent of |
|---|---|---|---|
| | | | Population |
| Resistance | Number of | | with |
| Score | Individuals | Phenotype | Phenotype |
| 9 | 37 | Tolerant | 46% |
| 8 | 8 | | |
| 7 | 2 | | |
| 6 | 4 | Intermediate | 11% |
| 5 | 5 | | |
| 4 | 2 | | |
| 3 | 12 | Susceptible | 43% |
| 2 | 20 | | |
| 1 | 13 | | |

Following phenotyping, the plants were leaf punched and the leaf tissue samples were freeze-dried in a lyophilizer for genotyping.

### Example 2

### Genotyping the Mapping Population

DNA was isolated from the collected leaf tissue using standard methods. A target region spanning nucleotide bases 1026370-1798401 of LG-O/Chrom. 10 (approximately 10 cM), which corresponds with a previously identified QTL for root-knot nematode (RKI) tolerance in soybean, was queried for SNPs (as described in Deschamps, et al. (2010) Plant Genome, 3(1):53-68) identified after Solexa resequencing of 61 soybean varieties. Additional SNPs were identified following Sanger-based resequencing of the QTL region in 25 putative R10 resistant lines and 23 putative susceptible lines. TaqMan® probes and related primers for two SNPs discovered via Sanger resequencing, S07181-1-Q2 and S07192-1-Q20, were designed and obtained, and TaqMan® SNP assays were administered as previously described (Livak, et al. (1995) Nat Genet., 9(4):341-2). Fourteen KASPar markers were also designed to the Solexa-derived SNPs using the Primerpicker software available from Kbiosciences and KASPar SNP assays were administered as previously described (Cuppen E. (2007), Genotyping by allele-specific amplification (KASPar) CSH Protoc. doi:10.1101/pdb.prot484).

The 16 markers were used to genotype the 107 individual plants in the mapping population. Eight of the 16 markers were determined to be monomorphic between the parental lines, and therefore these markers were excluded from subsequent analysis. The remaining eight polymorphic markers that were used in subsequent analyses are listed in Table 4, including the primer and probe sequences useful for detecting the markers. The estimated LG-O map position is based upon the genetic map of Choi, et al., (2007) Genetics 176:685-96. The SNP position on Ch 10 is based upon JGI Glymal assembly of the soybean physical map (Schmutz et al. (2010) Nature 463:178-183). In some examples, such as when KASPar detection chemistry is employed, allelic primers listed in Table 4 contain a 5′ tail sequence of GAAGGTGACCAAGTTCATGCT (SEQ ID NO: 27) when a FAM fluorescent label is used and a GAAGGTCGGAGTCAACGGATT (SEQ ID NO: 28) 5′ tail sequence when a VIC fluorescent label is used, with one of each of these tails/labels used on each of the allelic primers in the pair. The SNP allele present in the tolerant and susceptible parent for each marker is provided in Table 5.

TABLE 4
SNP markers associated with root-knot nematode tolerance

| | Estimated | Physical | | | |
|---|---|---|---|---|---|
| | LG-O Map | Position | | | |
| | Position | of SNP on | | | |
| | (cM) | Chrom. 10 | | | |
| | (based | (based | | | |
| | upon | upon JGI | Primer/Probe sequences | | |
| | Choi | Glyma1 | SEQ | | |
| Marker | 2007) | assembly) | ID: | Type | Sequence |
| S13251-1- | 6.2 | 1338884 | 1 | Allelic | CAATGTAGAACATTTTAGTTCACGAAATGT |
| K1 | | | | Primer | |
| | | | 2 | Allelic | CAATGTAGAACATTTTAGTTCACGAAATGA |
| | | | | Primer | |
| | | | 3 | Primer | TGGTATTTACAGCATCCCGCCGTA |
| S07191-1- | 7.8 | 1445721 | 4 | Primer | ATTTTCCAATTTTGCGTCAGA |
| Q2 | | | 5 | Primer | TTGGGAGTGAGATGTCTGGA |
| | | | 6 | Allelic | GAGCTGCTCTGCCAT |
| | | | | probe | |
| | | | 7 | Allelic | GAGCTGCTCTGCAAT |
| | | | | Probe | |
| S14256-1- | 8.1 | 1464172 | 8 | Allelic | CTAGACTAGTGGAGACTAAAAATGGT |
| K1 | | | | Primer | |
| | | | 9 | Allelic | CTAGACTAGTGGAGACTAAAAATGGG |
| | | | | Primer | |
| | | | 10 | Primer | CTAGGACTAAAGTGACAACAAATCACAATT |
| S07192-1- | 8.2 | 1470496 | 11 | Primer | CAACGTATTCTCGAACTAAAATGG |
| Q20 | | | 12 | Primer | TTGAATTAAGATCAAAGAGAAGAATTGA |
| | | | 13 | Allelic | CACCCACTTGATC |
| | | | | probe | |
| | | | 14 | Allelic | CCCACTTGTTCATC |
| | | | | Probe | |
| S14242-1- | 8.8 | 1506154 | 15 | Allelic | CATAAACACCTCCATTTCCAAAAGCAT |
| K1 | | | | Primer | |
| | | | 16 | Allelic | ATAAACACCTCCATTTCCAAAAGCAC |
| | | | | Primer | |
| | | | 17 | Primer | GTAGAGTGTTCCTTCGCATGTGGAT |
| S13261-1- | 9.5 | 1555737 | 18 | Allelic | GGACCTTTAATGTCATGTAAAAGATCCT |
| K1 | | | | Primer | |
| | | | 19 | Allelic | GACCTTTAATGTCATGTAAAAGATCCC |
| | | | | Primer | |
| | | | 20 | Primer | GCACAAAATATGCATAAAAGTAATGAGGAT |
| S14255-1- | 9.7 | 1567278 | 21 | Allelic | CCCAAGTGAAGGCTGAACCAG |
| K1 | | | | Primer | |
| | | | 22 | Allelic | ATCCCAAGTGAAGGCTGAACCAA |
| | | | | Primer | |
| | | | 23 | Primer | GTTGTTAGGCCACGCCCTACACTT |
| S14251-1- | 10.1 | 1593206 | 24 | Allelic | TAGCCAATTTTTGTGTCAACATTTTGTAG |
| K1 | | | | Primer | |
| | | | 25 | Allelic | AGCCAATTTTTGTGTCAACATTTTGTAC |
| | | | | Primer | |
| | | | 26 | Primer | CAAGGCAATTGTTGAGTCCGTCAAATTAA |

TABLE 5
Marker alleles in tolerant and susceptible lines

| | 513251- | S07191- | S14256- | S07192- | S14242- | S13261- | S14255- | S14251- |
|---|---|---|---|---|---|---|---|---|
| | 1-K1 | 1-Q2 | 1-K1 | 1-Q20 | 1-K1 | 1-K1 | 1-K1 | 1-K1 |
| Tolerant | T | G | A | T | A | G | C | C |
| allele | | | | | | | | |
| Susceptible | A | T | C | A | G | A | T | G |
| allele | | | | | | | | |

Map Manager QTX.b20 (Manly et al. (2001) Mammalian Genome 12: 930-932) was used to construct a linkage map and perform subsequent QTL analysis. The criteria for linkage evaluation was set to p=1e⁻⁵ and Kosambi mapping function was applied to convert the recombination fraction into map distance. The QTL effect was fit into an additive model. A 1000 permutation test was conducted to establish the threshold for statistical significance (LOD ratio statistic—LRS) to declare putative QTL. The mean phenotypic scores from each population were used for the QTL analysis.

Marker regression and interval mapping analysis completed using MapManager QTX.b20 indicated that the eight polymorphic SNPs are all tightly associated with the root-knot nematode tolerance trait (Likelihood Ratio Statistic: 155.7-202.9, Percent Variation Explained: 79-86%). Three of these SNPS, assayed by markers S07191-1-Q2, S14256-1-K1, S07192-1-Q20, were found to distinguish 20 varieties known to be tolerant to root-knot nematode infestation and 19 varieties known to be susceptible (see Table 6).

TABLE 6
Prediction of tolerant and susceptible
phenotypes using three markers

| Soybean | | | | |
|---|---|---|---|---|
| variety | Phenotype | S07191-1-Q2 | S14256-1-K1 | S07192-1-Q20 |
| 95Y20 | TOL | G | A | T |
| 95Y41 | TOL | G | A | T |
| 95M50 | TOL | G | A | T |
| 95M81 | TOL | G | A | T |
| 95Y70 | TOL | G | A | T |
| Gordon | TOL | G | A | N |
| Perrin | TOL | G | A | T |
| Govan | TOL | G | A | T |
| Forrest | TOL | G | A | T |
| Cobb | TOL | N | A | T |
| Walters | TOL | G | A | T |
| Hagood | TOL | G | A | T |
| Hartwig | TOL | G | A | T |
| Palmetto | TOL | G | A | T |
| Pharaoh | TOL | G | A | T |
| 97M50 | TOL | G | A | N |
| 95M30 | SUS | T | C | A |
| 94B73 | SUS | N | C | A |
| 94M80 | SUS | T | C | A |
| 94M90 | SUS | T | C | A |
| 95B43 | SUS | N | C | A |
| 95M82 | SUS | T | C | A |
| Hampton | SUS | T | C | A |
| Volstate | SUS | T | C | A |
| Johnston | SUS | T | C | A |
| S-100 | SUS | N | C | N |
| Narow | SUS | T | C | A |
| Hutcheson | SUS | T | C | A |
| Sharkey | SUS | T | C | A |
| Bossier | SUS | T | C | A |
| CNS | SUS | T | C | A |

The SNP markers identified in these studies could be useful, for example, for detecting and/or selecting soybean plants with improved tolerance to root-knot nematode. The physical position of each SNP is provided in Table 4 based upon the JGI Glymal assembly (Schmutz et al. (2010) Nature 463:178-183). Any marker capable of detecting a polymorphism at one of these physical positions, or a marker closely linked thereto, could also be useful, for example, for detecting and/or selecting soybean plants with improved root-knot nematode tolerance. In some examples, the SNP allele present in the tolerant parental line could be used as a favorable allele to detect or select plants with improved tolerance. In other examples, the SNP allele present in the susceptible parent line could be used as an unfavorable allele to detect or select plants without improved tolerance.

These SNP markers could also be used to determine a favorable or unfavorable haplotype. In certain examples, a favorable haplotype would include allele “T” for marker S13251-1-K1, allele “G” for marker S07191-1-Q2, allele “A” for marker S14256-1-K1, allele “T” for marker S07192-1-Q20, allele “A” for S14242-1-K1, allele “G” for marker S13261-1-K1, allele “C” for marker S14255-1-K1, and allele “C” for marker S14251-1-K1. In other examples, a favorable haplotype would include allele “G” for marker S07191-1-Q2, allele “A” for marker S14256-1-K1, and allele “T” for marker S07192-1-Q20. In addition to the markers listed in Table 4, other closely linked markers could also be useful for detecting and/or selecting soybean plants with improved root-knot nematode tolerance. Further, chromosome intervals containing the markers of Table 4 could also be used, such as the interval flanked by and including S13251-1-K1 and S14251-1-K1 on a physical map of LG-O. Other useful intervals include, for example the interval flanked by and including markers Sat_{—}132 and Satt500 on a genetic map of LG-O.

What is claimed is:

1. A method of detecting a first soybean plant or germplasm with improved root-knot nematode tolerance, the method comprising detecting at least one favorable allele of one or more marker locus selected from the group consisting of:
a) one or more markers on linkage group O selected from the group consisting of S13251-1-K1, S07191-1-Q2, S14256-1-K1, S07192-1-Q20, S14242-1-K1, S13261-1-K1, S14255-1-K1, and S14251-1-K1;
b) one or more markers within a genomic DNA region selected from the group consisting of SEQ ID NOs: 29, 31, 33, 35, 37, 39, 41, and 43;
c) one or more markers within an amplicon selected from the group consisting of SEQ ID NOs: 30, 32, 34, 36, 38, 40, 42, and 44;
d) one or more markers within a chromosome interval on linkage group O flanked by and including S13251-1-K1 and S14251-1-K1;
e) one or more markers within a chromosome interval on linkage group O flanked by and including S07191-1-Q2 and S07192-1-Q20;
f) one or more markers within a chromosome interval on linkage group O flanked by and including Sat_{—}132 and Satt500; and
g) one or more markers within a chromosome interval on chromosome 10 flanked by and including nucleotide positions 1338884 and 1593206.

2. The method of claim 1, wherein said detecting comprises detection of a SNP haplotype comprising two or more markers selected from the group consisting of S13251-1-K1, S07191-1-Q2, S14256-1-K1, S07192-1-Q20, S14242-1-K1, S13261-1-K1, S14255-1-K1, and S14251-1-K1.

3. The method of claim 1, wherein said detecting comprises detection of a SNP haplotype comprising four or more markers selected from the group consisting of S13251-1-K1, S07191-1-Q2, S14256-1-K1, S07192-1-Q20, S14242-1-K1, S13261-1-K1, S14255-1-K1, and S14251-1-K1.

4. The method of claim 1, wherein said detecting comprises detection of a SNP haplotype comprising six or more markers selected from the group consisting of S13251-1-K1, S07191-1-Q2, S14256-1-K1, S07192-1-Q20, S14242-1-K1, S13261-1-K1, S14255-1-K1, and S14251-1-K1.

5. The method of claim 1, wherein said detecting comprises detection of a SNP haplotype comprising markers S13251-1-K1, S07191-1-Q2, S14256-1-K1, S07192-1-Q20, S14242-1-K1, S13261-1-K1, S14255-1-K1, and S14251-1-K1.

6. The method of claim 1, wherein said detecting comprises detection of a SNP haplotype comprising markers S07191-1-Q2, S14256-1-K1, and S07192-1-Q20.

7. The method of claim 1, wherein said at least one favorable allele of one or more marker loci is selected from the group consisting of:
a) S13251-1-K1 allele T;
b) position 1338884 allele T;
c) S07191-1-Q2 allele G;
d) position 1445721 allele G;
e) S14256-1-K1 allele A;
f) position 1464172 allele A;
g) S07192-1-Q20 allele T;
h) position 1470496 allele T;
i) S14242-1-K1 allele A;
j) position 1506154 allele A;
k) S13261-1-K1 allele G;
l) position 1555737 allele G;
m) S14255-1-K1 allele C;
n) position 1567278 allele C;
o) S14251-1-K1 allele C; and
p) position 1593206 allele C.

8. The method of claim 5, wherein said SNP haplotype comprises the marker alleles
a) S13251-1-K1 allele T;
b) S07191-1-Q2 allele G;
c) S14256-1-K1 allele A;
d) S07192-1-Q20 allele T;
e) S14242-1-K1 allele A;
f) S13261-1-K1 allele G;
g) S14255-1-K1 allele C; and
h) S14251-1-K1 allele C.

9. The method of claim 6, wherein said SNP haplotype comprises the marker alleles
a) S07191-1-Q2 allele G;
b) S14256-1-K1 allele A; and
c) S07192-1-Q20 allele T.

10. The method of claim 1, wherein the detecting comprises amplifying the marker locus or a portion of the marker locus and detecting the resulting amplified marker amplicon.

11. The method of claim 10, wherein the amplifying comprises:
(1) admixing an amplification primer or amplification primer pair with a nucleic acid isolated from the first soybean plant or germplasm, wherein the primer or primer pair is complementary or partially complementary to at least a portion of the marker locus and is capable of initiating DNA polymerization by a DNA polymerase using the soybean nucleic acid as a template; and
(2) extending the primer or primer pair in a DNA polymerization reaction comprising a DNA polymerase and a template nucleic acid to generate at least one amplicon.

12. The method of claim 11, wherein the admixing of step 1) further comprises admixing at least one nucleic acid probe.

13. The method of claim 11, wherein the detection comprises PCR analysis.

14. The method of claim 1, further comprising selecting the first soybean plant or germplasm, or selecting a progeny of the first soybean plant or germplasm.

15. The method of claim 14, further comprising crossing the selected first soybean plant or germplasm with a second soybean plant or germplasm.

16. The method of claim 15, wherein the second soybean plant or germplasm comprises an exotic soybean strain or an elite soybean strain.

17. A plant selected by the method of claim 14.

18. A kit for selecting at least one soybean plant, the kit comprising:
a) primers or probes for detecting one or more marker loci associated with one or more quantitative trait loci associated with improved root-knot nematode tolerance, wherein the one or more marker loci are selected from the group consisting of:
i) one or more markers on linkage group O selected from the group consisting of S13251-1-K1, S07191-1-Q2, S14256-1-K1, S07192-1-Q20, S14242-1-K1, S13261-1-K1, S14255-1-K1, and S14251-1-K1;
ii) one or more markers within a genomic DNA region selected from the group consisting of SEQ ID NOs: 29, 31, 33, 35, 37, 39, 41, and 43;
iii) one or more markers within an amplicon selected from the group consisting of SEQ ID NOs: 30, 32, 34, 36, 38, 40, 42, and 44;
iv) one or more markers within the chromosome interval on linkage group O flanked by and including S13251-1-K1 and S14251-1-K1;
v) one or more markers within the chromosome interval on linkage group O flanked by and including S07191-1-Q2 and S07192-1-Q20;
vi) one or more markers within the chromosome interval on linkage group O flanked by and including Sat_{—}132 and Satt500; and
vii) one or more markers within the chromosome interval on chromosome 10 flanked by and including nucleotide positions 1338884 and 1593206; and
b) instructions for using the primers or probes for detecting the one or more marker loci and correlating the detected marker loci with predicted improved tolerance or increased susceptibility to root-knot nematode.

19. The kit of claim 18, wherein the primers or probes comprise one or more of SEQ ID NOs: 1-26 or 29-44.

20. The kit of claim 18, wherein the marker locus comprises S13251-1-K1 and wherein the primers or probes comprise SEQ ID NOs: 1-3.

21. The kit of claim 18, wherein the marker locus comprises S07191-1-Q2 and wherein the primers or probes comprise SEQ ID NOs: 4-7.

22. The kit of claim 18, wherein the marker locus comprises S14256-1-K1 and wherein the primers or probes comprise SEQ ID NOs: 8-10.

23. The kit of claim 18, wherein the marker locus comprises S07192-1-Q20 and wherein the primers or probes comprise SEQ ID NOs: 11-14.

24. The kit of claim 18, wherein the marker locus comprises S14242-1-K1 and wherein the primers or probes comprise SEQ ID NOs: 15-17.

25. The kit of claim 18, wherein the marker locus comprises S13261-1-K1 and wherein the primers or probes comprise SEQ ID NOs: 18-20.

26. The kit of claim 18, wherein the marker locus comprises S14255-1-K1 and wherein the primers or probes comprise SEQ ID NOs: 21-23.

27. The kit of claim 18, wherein the marker locus comprises S14251-1-K1 and wherein the primers or probes comprise SEQ ID NOs: 24-26.

28. An isolated polynucleotide capable of detecting a marker locus selected from the group consisting of S13251-1-K1, S07191-1-Q2, S14256-1-K1, S07192-1-Q20, S14242-1-K1, S13261-1-K1, S14255-1-K1, and S14251-1-K1.

29. The isolated polynucleotide of claim 28, wherein the polynucleotide comprises a nucleotide sequence selected from the group consisting of SEQ ID NOs: 1-26.

30. An isolated polynucleotide capable of detecting a nucleotide polymorphism on soybean chromosome 10 wherein the polymorphism is at a genomic location selected from the group consisting of position 1338884, position 1445721, position 1464172, position 1470496, position 1506154, position 1555737, position 1567278, and position 1593206.

31. A soybean plant with improved root knot nematode tolerance comprising at least one favorable allele of one or more marker locus selected from the group consisting of
a) one or more markers on linkage group O selected from the group consisting of S13251-1-K1, S07191-1-Q2, S14256-1-K1, S07192-1-Q20, S14242-1-K1, S13261-1-K1, S14255-1-K1, and S14251-1-K1;
b) one or more markers within a genomic DNA region selected from the group consisting of SEQ ID NOs: 29, 31, 33, 35, 37, 39, 41, and 43;
c) one or more markers within an amplicon selected from the group consisting of SEQ ID NOs: 30, 32, 34, 36, 38, 40, 42, and 44;
d) one or more markers within the chromosome interval on linkage group O flanked by and including S13251-1-K1 and S14251-1-K1;
e) one or more markers within the chromosome interval on linkage group O flanked by and including S07191-1-Q2 and S07192-1-Q20;
f) one or more markers within the chromosome interval on linkage group O flanked by and including Sat_{—}132 and Satt500; and
g) one or more markers within the chromosome interval on chromosome 10 flanked by and including nucleotide positions 1338884 and 1593206.

32. The plant of claim 31, wherein said plant comprises a SNP haplotype comprising markers S13251-1-K1, S07191-1-Q2, S14256-1-K1, S07192-1-Q20, S14242-1-K1, S13261-1-K1, S14255-1-K1, and S14251-1-K1.

33. The plant of claim 31, wherein said plant comprises a SNP haplotype comprising markers S07191-1-Q2, S14256-1-K1, and S07192-1-Q20.

34. The plant of claim 31, wherein said at least one favorable allele of one or more marker loci is selected from the group consisting of:
a) S13251-1-K1 allele T;
b) position 1338884 allele T;
c) S07191-1-Q2 allele G;
d) position 1445721 allele G;
e) S14256-1-K1 allele A;
f) position 1464172 allele A;
g) S07192-1-Q20 allele T;
h) position 1470496 allele T;
i) S14242-1-K1 allele A;
j) position 1506154 allele A;
k) S13261-1-K1 allele G;
l) position 1555737 allele G;
m) S14255-1-K1 allele C;
n) position 1567278 allele C;
o) S14251-1-K1 allele C; and
p) position 1593206 allele C.

35. The plant of claim 32, wherein said SNP haplotype comprises the marker alleles:
a) S13251-1-K1 allele T;
b) S07191-1-Q2 allele G;
c) S14256-1-K1 allele A;
d) S07192-1-Q20 allele T;
e) S14242-1-K1 allele A;
f) S13261-1-K1 allele G;
g) S14255-1-K1 allele C; and
h) S14251-1-K1 allele C.

36. The plant of claim 33, wherein said SNP haplotype comprises the marker alleles:
a) S07191-1-Q2 allele G;
b) S14256-1-K1 allele A; and
c) S07192-1-Q20 allele T.

37. The soybean plant of claim 31, wherein said plant is an elite soybean variety.
